(19) United States
(12) Patent Application Publication
(10) Pub. No.: US 2015/0110672 A1
(43) Pub. Date: Apr. 23, 2015

(54) METHOD FOR INDUCING APOPTOSIS OF DISEASE CELLS AND DISEASE-CAUSING MICROORGANISMS USING PLASMA FOR BIO-MEDICAL USE

(71) Applicant: Ajou University Industry-Academic Cooperation Foundation, Suwon (KR)

(72) Inventors: Sang Sik Yang, Seoul (KR); Eun Pyo Moon, Seoul (KR); Jong Soo Lee, Seoul (KR); Kang Il Kim, Suwon (KR)

(21) Appl. No.: 14/472,863

(22) Filed: Aug. 29, 2014

Related U.S. Application Data
(63) Continuation of application No. PCT/KR2013/001438, filed on Feb. 22, 2013.

(30) Foreign Application Priority Data
Feb. 29, 2012 (KR) 10-2012-0021092

(51) Int. Cl. A61L 2/18

(52) U.S. Cl. CPC A61L 2/18
USPC 422/29

(57) ABSTRACT

The present invention relates to a method for inducing apoptosis of disease cells and disease-causing micro-organisms using plasma for bio-medical use, the method comprising the steps of: generating plasma by means of a high-pressure plasma jet produced using a micro-electro-mechanical system (MEMS) technique; plasma-processing by applying a solution with the plasma; exposing cells to the plasma-processed solution; and inactivating disease cells and disease-causing micro-organisms from among the exposed cells. According to the present invention, after applying plasma to a solution such as a buffer solution or water, targets to be processed such as micro-organisms or animal or plant cells are exposed thereto, and thus the present invention can be used in both bio and medical fields, and has the benefit of effectively inducing apoptosis of disease cells and disease-causing micro-organisms with low power due to indirect processing.

### CROSS-REFERENCE TO RELATED APPLICATION

This application is a continuation of International Application No. PCT/KR2013/001438 filed on Feb. 22, 2013, which claims priority to Korean Application No. 10-2012-0021092 filed on Feb. 29, 2012, which applications are incorporated herein by reference.

### TECHNICAL FIELD

The present invention relates to a method of inducing the apoptosis of disease cells and disease-causing microorganisms using plasma for the purpose of bio-medical application and, more particularly, to a method of inducing the apoptosis of disease cells and disease-causing microorganisms using plasma for the purpose of bio-medical application, which is capable of inducing the apoptosis of abnormal cells, such as disease cells and disease-causing microorganisms, using plasma.

### BACKGROUND ART

Recently, the trend of plasma application programs has been directed to the bioscience field. Representative bio-medical plasma application programs are classified into the coagulation of blood, the apoptosis of cancer cells, and collaborative dental treatment.

In this case, plasma that is used in bio-medical application programs should be generated under low-temperature and atmospheric-pressure conditions. If these conditions are not met, biological substances may be damaged by plasma.

Technology for eliminating microorganisms using plasma is proposed in Korean Patent Application Publication Nos. 10-2011-0006017 and 10-2003-0060644.

As conventional technologies, supporting structures disclosed in Korean Patent Application Publication Nos. 10-2011-0006017 and 10-2003-0060644 are briefly described below.

FIG. 1 is a block diagram conceptually illustrating the configuration of an atmospheric-pressure low-temperature air plasma generation apparatus disclosed in Korean Patent Application Publication No. 10-2011-0006017 (hereinafter referred to as “conventional technology 1”). Referring to FIG. 1, a method of eliminating microorganisms using air plasma emitted from a porous dielectric inserted between electrodes in accordance with conventional technology 1 is performed via an atmospheric-pressure low-temperature air plasma generation apparatus, including a power source 10, a power supply unit 20, an air supply unit 30, a reactor 40, and a jet discharge unit 50. This method of eliminating microorganisms by generating air plasma in a reactor including electrodes between which a porous dielectric has been inserted includes constructing a plasma reactor including a power electrode and a ground electrode; inserting a porous dielectric between the power electrode and the ground electrode; applying an insulator onto the surface of the power electrode directed toward the ground electrode; injecting air into the plasma reactor; supplying medium-frequency and low-frequency power to the power electrode, thereby inducing the discharging of the porous dielectric and generating plasma; discharging the generated plasma via the jet discharge unit in the form of plasma jets by always injecting sufficient air into the reactor; and eliminating microorganisms using the plasma jets.

However, since the method of eliminating microorganisms using air plasma emitted from a porous dielectric inserted between electrodes in accordance with conventional technology 1 directly eliminates microorganisms attached on the surface of an object by spraying air plasma, it is problematic in that the concentration of plasma should be kept uniform to achieve the uniformity of elimination.

FIG. 2 is a flowchart illustrating a sterilization procedure disclosed in Korean Patent Application Publication No. 10-2003-0060644 (hereinafter referred to as “conventional technology 2”).

A sterilization method using plasma under atmospheric pressure in accordance with conventional technology 2 supplies high-frequency power between two electrodes by operating a power supply device (102 of FIG. 1) in order to generate atmospheric-pressure plasma and then injects reaction gas at steps S1 and S2. When the reaction gas enters into a space between the two electrodes to which high voltage has been applied, atmospheric-pressure plasma is generated by dielectric layer discharge and then an object to be processed is sterilized and detoxified using the plasma at steps S3 and S4.

Meanwhile, a sterilization and detoxification method according to the present invention includes a method of bringing an object to be processed into direct contact with ions, electrons and active radicals generated in the reaction area of a plasma generation apparatus (see FIG. 2), and a method of spraying plasma generated in a reaction area using gas pressure and a spray tube and transferring the plasma to an object to sterilized and detoxified (see FIG. 3). In this case, when a Helmholtz coil or solenoid coil for generating a magnetic field is installed in a generation tube or a chamber, plasma can be effectively transferred to an object to be processed, and thus sterilization and detoxification ability can be improved.

Furthermore, gaseous water H₂O, hydrogen peroxide H₂O₂, alcohol, acetone, argon, hydrogen, helium, oxygen, compressed air, etc. may be used as reaction gas injected into a plasma generation chamber or tube via the gas injection tube of a gas supply device 104. These reaction gases generate atmospheric-pressure plasma through dielectric layer discharge when AC power ranging from hundreds of volts (V) to tens of kilovolts (KV) is applied between two electrodes in an atmospheric-pressure state. In this case, in a plasma generation area, ions, electrons, active radicals and ozone are generated. In particular, as water, hydrogen peroxide, alcohol, acetone, etc. are resolved in a discharge area, a large number of active radicals having excellent oxidizing capability are generated, and thus effectively eliminate germs and toxic substances on the surface of an object to be processed within a few seconds or minutes.

However, the sterilization method using plasma under atmospheric pressure in accordance with conventional technology 2 is problematic in that separate equipment for keeping the concentration of plasma uniform in the process of eliminating disease-causing microorganisms by spraying atmospheric-pressure plasma should be provided.

### SUMMARY OF THE DISCLOSURE

The present invention has been made keeping in mind the above problems occurring in the prior art, and an object of the present invention is to provide a method of inducing the apoptosis of disease cells and disease-causing microorganisms using plasma for the purpose of bio-medical application, in which a solution, such as a buffer solution or water, is irradiated with plasma and then processing targets, such as microorganisms or animal or plant cells, are processed by exposing the targets to the solution, thereby being able to be applied to both the biological and medical fields and also being able to efficiently induce the apoptosis of disease cells and disease-causing microorganisms using low power through indirect processing.

Furthermore, another object of the present invention is to provide a method of inducing the apoptosis of disease cells and disease-causing microorganisms using plasma for the purpose of bio-medical application, in which processing targets, such as microorganisms or animal or plant cells, are processed by exposing the targets directly to plasma, thereby being able to perform sterilization and induce the apoptosis of disease cells and disease-causing microorganisms using various methods, such as direct processing.

In order to achieve the above objects, the present invention provides a method of inducing the apoptosis of disease cells and disease-causing microorganisms using plasma for the purpose of bio-medical application, including generating plasma via an atmospheric-pressure plasma jet fabricated using microelectromechanical systems (MEMS) technology; plasma-processing a solution by irradiating the solution with the plasma; exposing cells to the plasma-processed solution; and inactivating disease cells and disease-causing microorganisms of the cells exposed to the solution.

The cells may be microorganisms or animal or plant cells.

The microorganisms may be bacteria.

The solution may be a buffer solution or water.

The present invention has the advantages of being able to be applied to both the biological and medical fields and also being able to efficiently induce the apoptosis of disease cells and disease-causing microorganisms using low power through indirect processing because a solution, such as a buffer solution or water, is irradiated with plasma and then processing targets, such as microorganisms or animal or plant cells, are processed by exposing the targets to the solution.

Furthermore, the present invention has the advantage of being able to perform sterilization and induce the apoptosis of disease cells and disease-causing microorganisms using various methods, such as direct processing, because processing targets, such as microorganisms or animal or plant cells, are processed by exposing the targets directly to plasma.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a block diagram conceptually illustrating the configuration of an atmospheric-pressure low-temperature air plasma generation apparatus in accordance with conventional technology 1;

FIG. 2 is a flowchart illustrating a sterilization procedure in accordance with conventional technology 2;

FIG. 3 is a flowchart of a method of inducing the apoptosis of disease cells and disease-causing microorganisms using plasma for the purpose of bio-medical application in accordance with the present invention;

FIG. 4 is a schematic diagram of an atmospheric-pressure plasma jet for generating plasma that is used in the method of inducing the apoptosis of disease cells and disease-causing microorganisms using plasma for the purpose of bio-medical application in accordance with the present invention;

FIGS. 5A, 5B, 5C, and 5D show graphs of voltages and currents that were measured when specific voltages were applied in the method of inducing the apoptosis of disease cells and disease-causing microorganisms using plasma for the purpose of bio-medical application in accordance with the present invention are illustrated; and

FIG. 6A illustrates the state in which the voltage applied to the atmospheric-pressure plasma jet 10 has been cut off. FIG. 6B illustrates the state in which the voltage applied to the atmospheric-pressure plasma jet 10 is 5.5 kVp-p. FIG. 6C illustrates the state in which the voltage applied to the atmospheric-pressure plasma jet 10 is 7.5 kVp-p. FIG. 6D illustrates the state in which the voltage applied to the atmospheric-pressure plasma jet 10 is 9.5 kVp-p.

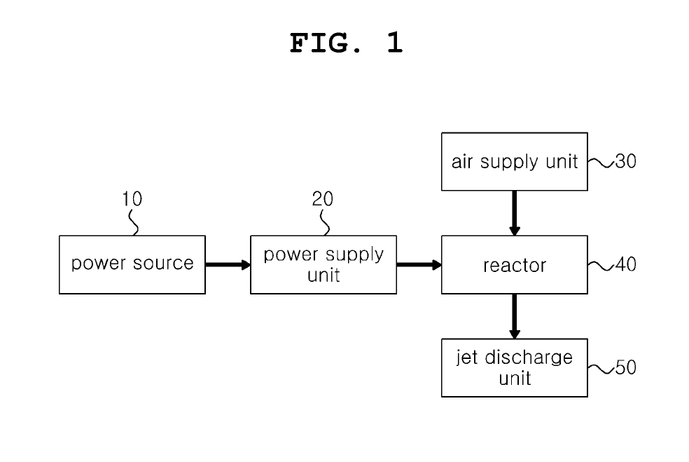

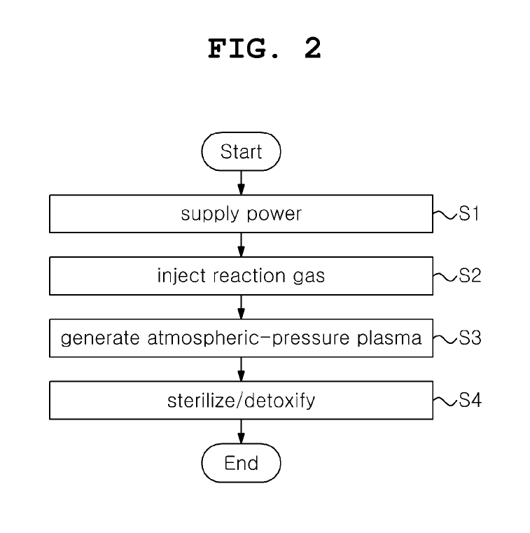

### DETAILED DESCRIPTION OF THE DISCLOSURE

In order to achieve the above objects, the present invention provides a method of inducing the apoptosis of disease cells and disease-causing microorganisms using plasma for the purpose of bio-medical application, including generating plasma via an atmospheric-pressure plasma jet fabricated using microelectromechanical systems (MEMS) technology; plasma-processing a solution by irradiating the solution with the plasma; exposing cells to the plasma-processed solution; and inactivating disease cells and disease-causing microorganisms of the cells exposed to the solution.

The cells may be microorganisms or animal or plant cells.

The microorganisms may be bacteria.

The solution may be a buffer solution or water.

### MODE FOR INVENTION

The terms and words used in the present specification and the claims should be interpreted as having meanings and concepts appropriate for the technical concept of the present invention based on the principle in which an inventor can appropriately define the concepts of terms in order to describe his or her invention in the best way.

Throughout the specification, when any part is described as “comprising” or “including” any component, this means that the part does not exclude any other component but includes any other component, unless described otherwise. The term “unit” described in the specification means a unit for processing at least one function or operation, and may be implemented as hardware, software, or combinations thereof.

The configuration of an embodiment of a method of inducing the apoptosis of disease cells and disease-causing microorganisms using plasma for the purpose of bio-medical application in accordance with the present invention will be described in detail with reference to the drawings.

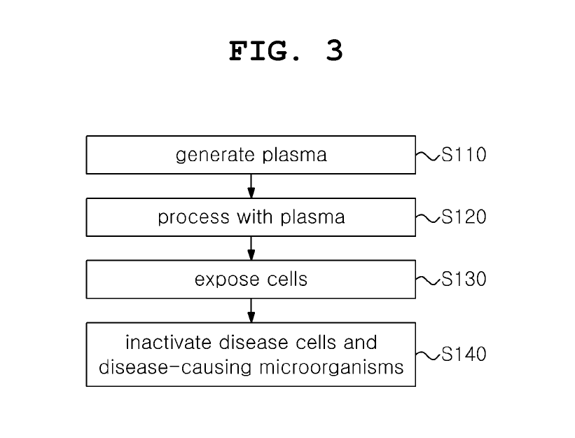

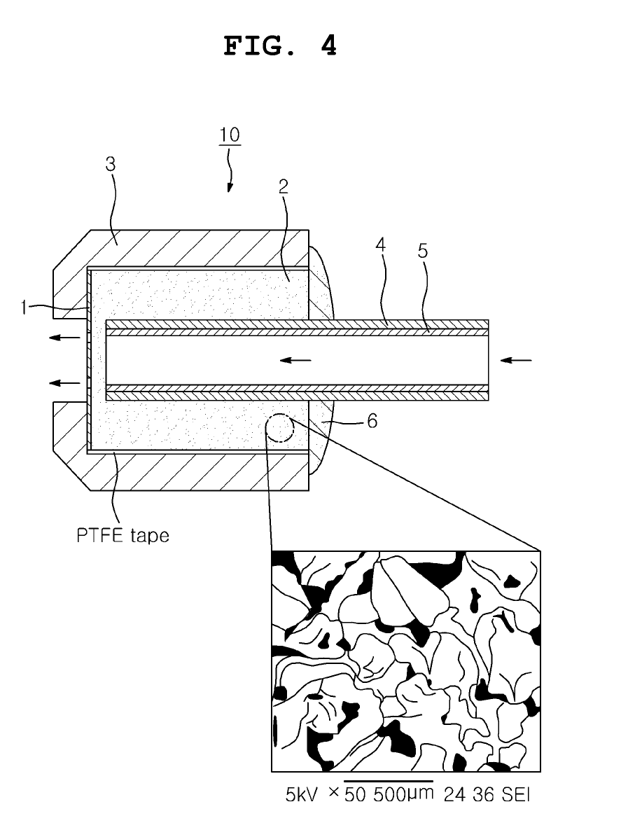

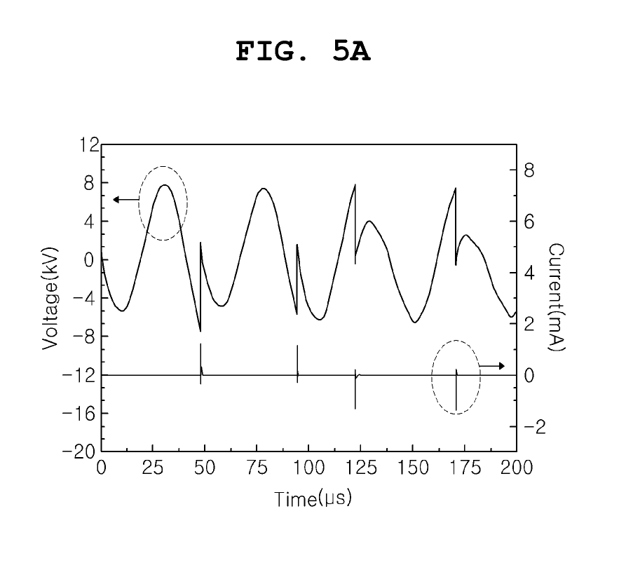

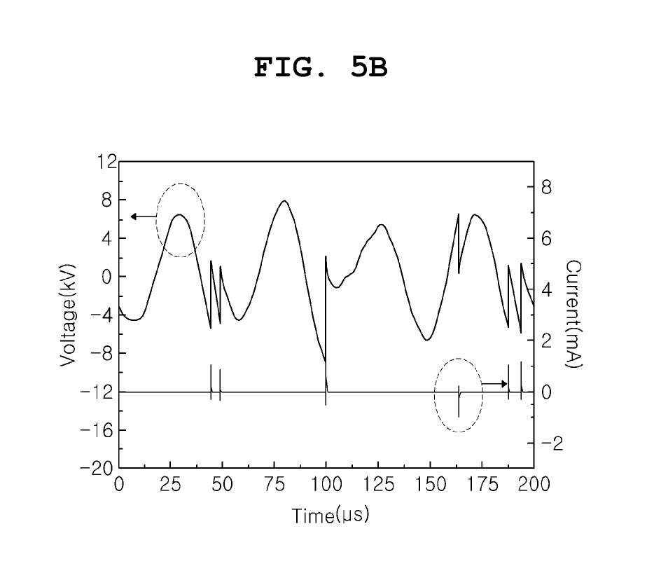

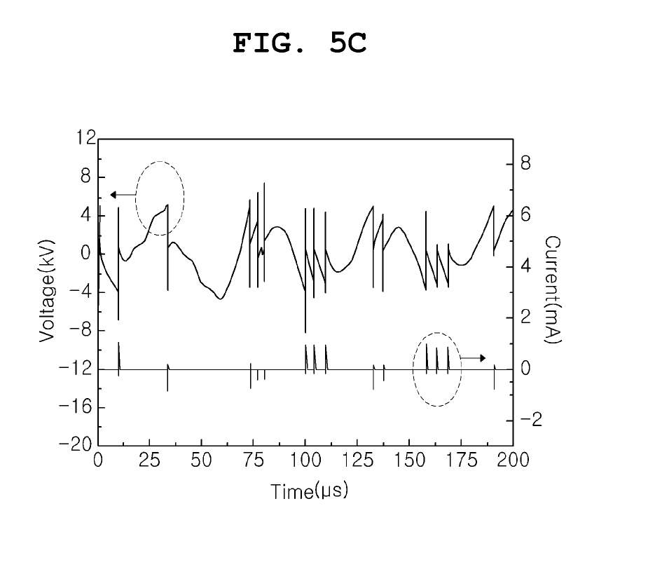

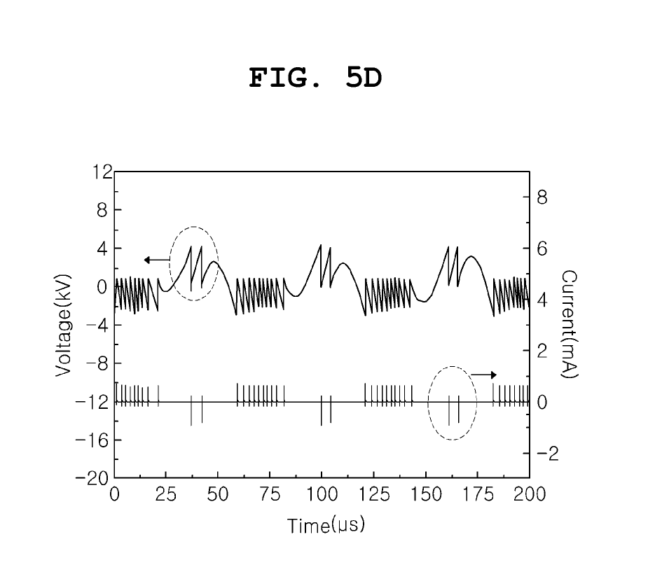

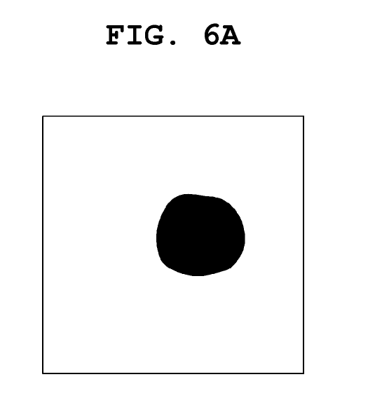

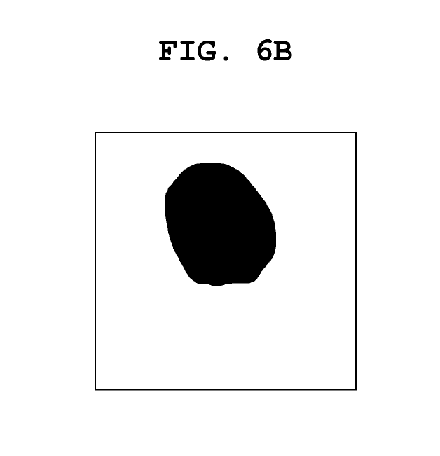

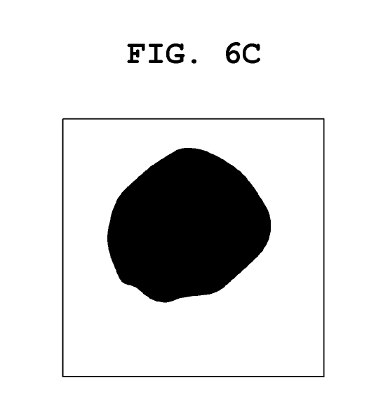

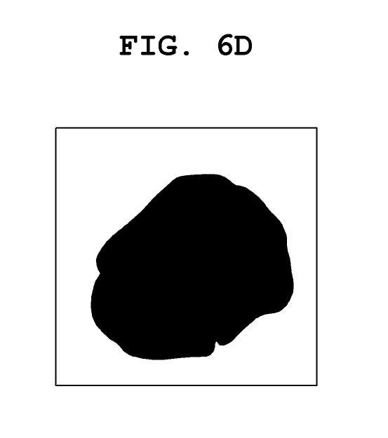

In FIG. 3, a method of inducing the apoptosis of disease cells and disease-causing microorganisms using plasma for the purpose of bio-medical application in accordance with the present invention is illustrated as a flowchart; in FIG. 4, an atmospheric-pressure plasma jet for generating plasma that is used in the method of inducing the apoptosis of disease cells and disease-causing microorganisms using plasma for the purpose of bio-medical application in accordance with the present invention is illustrated as a schematic diagram; in FIGS. 5A, 5B, 5C and 5D, graphs of voltages and currents that were measured when specific voltages were applied in the method of inducing the apoptosis of disease cells and disease-causing microorganisms using plasma for the purpose of bio-medical application in accordance with the present invention are illustrated; and in FIGS. 6A, 6B, 6C and 6D, photos that are used to estimate the density of plasma by irradiating red ink, dropped onto a slide glass, with plasma in the method of inducing the apoptosis of disease cells and disease-causing microorganisms using plasma for the purpose of bio-medical application in accordance with the present invention.

In accordance with these drawings, a method of inducing the apoptosis of disease cells and disease-causing microorganisms using plasma for the purpose of bio-medical application in accordance with the present invention includes a plasma generation step S100, a plasma irradiation step S110, a cell exposure step S120, and a disease cells and disease-causing microorganisms inactivation step S130.

The plasma generation step S100 is the step of generating plasma via an atmospheric-pressure plasma jet fabricated using microelectromechanical systems (MEMS) technology.

In this case, the structure of the atmospheric-pressure plasma jet 10 for generating plasma is described, as follows:

The atmospheric-pressure plasma jet 10 includes an electrode 1 used as an anode, a porous insulation material 2, a casing 3, a protective tube 4, a gas injection tube 5 used as a cathode, and a sealant 6.

The electrode 1 used as an anode is formed in the shape of a disk, and a plurality of holes is formed in the center portion of the electrode 1 so that the plurality of holes is located within a hole formed in the front center portion of the casing 3. The plasma generated between the electrode 1, that is, an anode, and the gas injection tube 5, that is, a cathode, by discharge is sprayed through the plurality of holes. Meanwhile, in this embodiment, the electrode 1 is preferably made of metal, more preferably nickel.

The front surface of the porous insulation material 2 comes into tight contact with the rear surface of the electrode 1 and the rear portion of the porous insulation material 2 is formed in the shape of an open cylinder. Accordingly, the porous insulation material 2 insulates the electrode 1 and the gas injection tube 5 while surrounding the outer circumferential surface of the gas injection tube 5 inserted into the open rear portion of the porous insulation material 2.

Furthermore, the porous insulation material 2 preferably has a large number of pores to achieve transmission because the porous insulation material 2 transmits gas, injected from the gas injection tube 5, to the electrode 1. In this case, in this embodiment, the porous insulation material 2 is preferably made of a ceramic material, more preferably an alumina material. In this case, a discharge is generated to generate plasma between the electrode 1 and the gas injection tube 5. The porous insulation material 2 prevents such a discharge from spreading to the outside.

The casing 3 surrounds the outer circumferential surface of the porous insulation material 2 surrounding the outer portion of the electrode 1 and the front portion of the gas injection tube 5, and is made of an aluminum material.

Furthermore, since persistent high-temperature heat resistance is required between the outer circumferential surface of the porous insulation material 2 and the inner circumferential surface of the casing 3, a PTFE tape is applied in order to prevent incoming gas from leaking.

The protective tube 4 surrounds the outer circumferential surface of the gas injection tube 5, thereby performing the function of insulating and protecting the gas injection tube 5 from the outside. In this embodiment, the protective tube 4 is preferably made of a quartz material.

The gas injection tube 5 connects to and communicates with a process gas injection tube (not illustrated in the drawings) so that gas can be injected from the outside. In this embodiment, the gas injection tube 5 is preferably made of a stainless steel material.

The sealant 6 is Torr Seal that surrounds the rear surfaces of the porous insulation material 2 and the casing 3, and the exposed portion of the gas injection tube 5 close to the rear surface of the porous insulation material 2, and seals off the gap between the contact surfaces of the porous insulation material 2 and the casing 3 and the gap between the connection surfaces of the porous insulation material 2 and the gas injection tube 5.

The principle by which the above-described atmospheric-pressure plasma jet 10 generates and sprays plasma is as follows. The gas entering via the gas injection tube 5 is ionized by an electric field generated between the holes of the electrode 1 and the gas injection tube 5 while passing through the porous insulation material 2, in which manner plasma is generated. The plasma generated as described above is sprayed through the holes of the electrode 1 by being pushed by the gas entering via the gas injection tube 5.

For example, plasma discharge tests using the atmospheric-pressure plasma jet 10 are conducted using nitrogen N₂ gas under atmospheric pressure, in which case the flow rate of gas entering into the atmospheric-pressure plasma jet 10 is 4 L/min and alternating current (AC) power is used as applied power.

That is, in order to investigate the electrical characteristics of discharge based on the applied voltage of the atmospheric-pressure plasma jet 10, tests were conducted with the flow rate of input gas fixed to 4 L/min and applied voltage varied. It could be observed that, when the voltage applied to the atmospheric-pressure plasma jet 10 was 3.5 kVp-p, discharge started, but plasma was not sprayed from the anode hole of the atmospheric-pressure plasma jet 10 to the air and the discharge was unstable. In order to investigate the electrical characteristics of plasma, when the voltage applied to the atmospheric-pressure plasma jet 10 was 3.5 kVp-p, 5.5 kVp-p, 7.5 kVp-p, and 9.5 kVp-p, voltage and current were measured.

FIGS. FIGS. 5A, 5B, 5C and 5D illustrate the voltage and current measured at each applied voltage. In FIGS. 5A, 5B, 5C and 5D, saw tooth-shaped, rapidly decreasing voltage and rapidly increasing current waveforms indicate micro-discharge that occurs when discharge is generated along the pores of alumina, in which case plasma is generated and sprayed. As can be seen from the graph, as the applied voltage increased, the number of micro-discharges increased. In particular, at 9.5 kVp-p, it can be seen that stable discharge occurred periodically. From this result, it is expected that as the applied power increases, the number of micro-discharges increases, and the density of sprayed plasma increases. However, at the applied voltage equal to or higher than 9.5 kVp-p, when discharge time was long, a phenomenon occurred in which plasma was sprayed through all the holes, become unstable and was then spayed through only a single hole temperature due to an increase in the temperature of the atmospheric-pressure plasma jet 10. In this case, it was found after discharge that during the discharge, sputtering occurred while plasma was being sprayed through the holes of the anode, damage occurred, and thus an one-side hole become widened. Accordingly, it can be seen that there is the upper limit value of the applied voltage. In order to prevent damage to the electrode while spraying stable plasma, the voltage applied to the atmospheric-pressure plasma jet 10 should be equal to or higher than 5.5 kVp-p and equal to lower than 9.5 kVp-p.

In particular, in FIG. 5D, as a large number of pulses occurred at about 3.11 kV due to a gradual increase in input voltage, successive decreases in the effective value Vrms appeared, and an increase of about 0.77 mA in current appeared due to an increase in average discharge current. In this case, the atmospheric-pressure plasma jet 10 wasted a small power of about 2.4 W compared to the output of plasma.

FIGS. 6A, 6B, 6C, and 6D illustrate photos that are used to estimate the density of plasma by irradiating red ink, dropped onto a slide glass, with plasma. FIG. 6A illustrates the state in which the voltage applied to the atmospheric-pressure plasma jet 10 has been cut off, FIG. 6B illustrates the state in which the voltage applied to the atmospheric-pressure plasma jet 10 is 5.5 kVp-p, FIG. 6C illustrates the state in which the voltage applied to the atmospheric-pressure plasma jet 10 is 7.5 kVp-p, and FIG. 6D illustrates the state in which the voltage applied to the atmospheric-pressure plasma jet 10 is 9.5 kVp-p.

In this case, with regard to test conditions, the distance between the atmospheric-pressure plasma jet 10 and the slide glass was 1 cm, and plasma processing time was 10 sec. From these results, it can be seen that the hydrophilic property of the surface of the red ink increases with an increase in the applied voltage, which is the same as the electrical characteristics of the plasma.

Meanwhile, at the plasma generation step S100, it may be possible to adjust the state of the plasma through the control of conditions, including the intensity of the plasma, the amount of output of the plasma, the type of gas and the flow rate of the gas, upon generating the plasma.

The plasma irradiation step S110 is the step of exposing cells to the plasma-processed solution. A buffer solution or water is used as the solution. Furthermore, the microorganisms of the cells as which microorganisms or animal or plant cells are used are bacteria including Pectobacterium carotovorum or Staphylococcus aureus.

Meanwhile, in the method of inducing the apoptosis of disease cells and disease-causing microorganisms using plasma for the purpose of bio-medical application in accordance with the present invention, targets whose apoptosis is induced are microorganisms or animal or plant cells, as described above. In this case, microorganism processing methods include plasma processing methods and processing methods using plasma-processed microorganisms.

The plasma processing methods are divided into direct processing methods and an indirect processing method. The direct processing methods are classified into a method of processing microorganisms on the surface of a plant with plasma generated by the atmospheric-pressure plasma jet 10, and a method of processing microorganisms on a solid medium, that is, a culture medium, with plasma generated by the atmospheric-pressure plasma jet 10. In this case, the solid medium is a medium formed by solidifying bouillon with agar (sometimes gelatin). A medium formed by solidifying serum by heating it is used as the solid medium depending on its purpose.

Furthermore, the indirect processing method is a method of processing phosphate buffer saline (PBS), that is, a kind of buffer solution, with plasma generated by the atmospheric-pressure plasma jet 10 and then mixing the plasma-processed PBS with microorganisms.

With regard to the processing methods using plasma-processed microorganisms, Pectobacterium carotovorum or Staphylococcus aureus, that is, a kind of bacteria, is processed with plasma generated by the atmospheric-pressure plasma jet 10, and then the plasma-processed Pectobacterium carotovorum, that is, plasma-processed microorganisms, may be applied to both the direct and indirect processing methods of the above-described plasma processing methods. In contrast, the plasma-processed Staphylococcus aureus, that is, plasma-processed microorganisms, does not cause a disease to plants, and thus the plasma-processed Staphylococcus aureus may be used for the method of processing microorganisms on a solid medium, that is, the direct processing methods of the plasma processing methods, except for the method of processing microorganisms on the surface of a plant with plasma generated by the atmospheric-pressure plasma jet 10, and for the method of processing a buffer solution and then mixing the buffer solution with microorganisms.

The cell exposure step S120 is the step of exposing cells to the solution processed via the plasma generated by the atmospheric-pressure plasma jet 10.

The disease cells and disease-causing microorganisms inactivation step S130 is the step of inactivating the disease cells and disease-causing microorganisms of the cells exposed to the plasma-processed solution.

Although the present invention has been described above based on the limited embodiments and diagrams, the present invention is not limited to these embodiments, but a person having ordinary knowledge in the technical field to which the present invention pertains can make various modifications and variations from the above description.

Therefore, the scope of the present invention should not be defined based on only the described embodiments, but should be defined based on not only the following claims but also equivalents to these claims.

### INDUSTRIAL APPLICABILITY

The present invention relates to a method of inducing the apoptosis of disease cells and disease-causing microorganisms using plasma for the purpose of bio-medical application, and the present invention includes generating plasma via an atmospheric-pressure plasma jet fabricated using microelectromechanical systems (MEMS) technology; plasma-processing a solution by irradiating the solution with the plasma; exposing cells to the plasma-processed solution; and inactivating disease cells and disease-causing microorganisms of the cells exposed to the solution.

The present invention has the advantages of being able to be applied to both the biological and medical fields and also being able to efficiently induce the apoptosis of disease cells and disease-causing microorganisms using low power through indirect processing because a solution, such as a buffer solution or water, is irradiated with plasma and then processing targets, such as microorganisms or animal or plant cells, are processed by exposing the targets to the solution.

What is claimed is:

1. A method of inducing the apoptosis of disease cells and disease-causing microorganisms using plasma for a purpose of bio-medical application, comprising:
generating plasma via an atmospheric-pressure plasma jet fabricated using microelectromechanical systems (MEMS) technology;
plasma-processing a solution by irradiating the solution with the plasma;
exposing cells to the plasma-processed solution; and
inactivating disease cells and disease-causing microorganisms of the cells exposed to the solution.

2. The method of claim 1, wherein the cells are microorganisms or animal or plant cells.

3. The method of claim 2, wherein the microorganisms are bacteria.

4. The method of claim 1, wherein the solution is a buffer solution or water.
